(19) United States
(12) Patent Application Publication
(10) Pub. No.: US 2019/0320960 A1
(43) Pub. Date: Oct. 24, 2019

(54) BLOOD COLLECTION DEVICE WITH INTEGRATED ABSORBENT MATERIAL

(71) Applicant: SIEMENS HEALTHCARE DIAGNOSTICS INC., TARRYTOWN, NY (US)

(72) Inventors: Eric Olson, North Salem, NY (US); Steven Bellofatto, Closter, NJ (US); Steven Madsen, New York, NY (US); Courtney Nicholls, Cranston, RI (US)

(21) Appl. No.: 16/349,916

(22) PCT Filed: Nov. 14, 2017
(86) PCT No.: PCT/US17/61592

§ 371 Date: May 14, 2019

Related U.S. Application Data
(60) Provisional application No. 62/421,844, filed on Nov. 14, 2016.

(51) Int. Cl. A61B 5/15; A61B 5/151

(52) U.S. Cl. CPC A61B 5/150343; A61B 5/150022; A61B 5/150351; A61B 5/15115; A61B 5/150977; A61B 5/150419

(57) ABSTRACT

A blood collection device includes a housing and an interface. The blood collection device includes at least one blood collection container engaged with the interface and at least one lancet coupled to the interface. The at least one lancet extends or is configured to extend from the interface. The at least one lancet is configured to pierce tissue. The blood collection device also includes an absorbent material integrated with the housing. The absorbent material is configured to absorb fluids emanating from the tissue after the at least one lancet pierces the tissue.

### CROSS-REFERENCE TO RELATED APPLICATIONS

This application claims priority to and the benefit of U.S. Provisional Application Ser. No. 62/421,844, filed Nov. 14, 2016, the entire contents of which are incorporated by reference into the present application.

### TECHNICAL FIELD

The present disclosure is directed to a blood collection device with an integrated absorbent material.

### BACKGROUND

When collecting blood for use in clinical diagnostics, medical professionals must follow a complex procedure with many steps. The steps require the manual manipulation of many small implements such as tubes, needles, wipes, and gauze, so there is a significant amount of manual dexterity required. Because there are a large number of steps, it is possible that the person collecting blood, i.e. a collection technician will accidentally skip a step or perform steps in the incorrect order. Innovations that reduce the number of steps required to collect blood reduce the likelihood of error. Because the steps require the manual manipulation of many small implements, it is possible that the collection tech will accidentally drop or misplace something, which can cause errors in diagnosis and risk of infection. Innovations that reduce the amount of manual dexterity required to collect blood reduce the likelihood of error.

In some cases, multiple tubes of blood are required. This is often the case because different types of analyses require the use of specimen containers with different types of additives used to stabilize the blood. In these cases, several steps of the procedure for collecting blood are repeated, compounding the opportunity for error. For example, when collecting blood through venipuncture (or with a lancet), the collection technician can reuse the same needle for subsequent tubes but several steps must be repeated for each additional tube. In addition, each tube must be inverted shortly after collection in order to ensure proper mixing of any additives in the tube. The inversion step is often done in parallel while the next tube is filling. Because of this multitasking, it is possible to forget the inversion step for the prior tube, or to drop something while filling one tube with one hand and inverting another tube with the other hand. In some cases, the collection technician must apply gauze to the wound immediately after collection is complete. The gauze application step is often done in parallel while inverting the prior tube. Because of this multitasking, it is possible to forget the inversion step for the prior tube, or to drop something while applying gauze with one hand and inverting a tube with the other hand.

### SUMMARY

An embodiment of the present disclosure includes a blood collection device. The blood collection device includes a housing and an interface. The blood collection device includes at least one blood collection container engaged with the interface and at least one lancet coupled to the interface. The at least one lancet extends or is configured to extend from the interface. The at least one lancet is configured to pierce tissue. The blood collection device also includes an absorbent material integrated with the housing. The absorbent material is configured to absorb fluids emanating from the tissue after the at least one lancet pierces the tissue.

### BRIEF DESCRIPTION OF THE DRAWINGS

The foregoing summary, as well as the following detailed description, will be better understood when read in conjunction with the appended drawings. The drawings show illustrative embodiments of the disclosure. It should be understood, however, that the application is not limited to the precise arrangements and instrumentalities shown.

FIG. 1 is a front elevation view of the blood collection device according to an embodiment of the present disclosure;

FIG. 2 is a side partial sectional view of the blood collection device illustrated in FIG. 1;

FIG. 3 is a side partial sectional view of the blood collection device according to another embodiment of the present disclosure;

FIG. 4 is a perspective view of a blood collection device with a cap removed according to another embodiment of the present disclosure;

FIG. 5 is a side view of the blood collection device illustrated in FIG. 4;

FIG. 6 is a front elevation view of the blood collection device shown in FIG. 4;

FIG. 7 is a front elevation view of the blood collection device according to another embodiment of the present disclosure;

FIG. 8 is a front elevation view of the blood collection device according to another embodiment of the present disclosure; and

FIG. 9 is a side view of the blood collection container according to an embodiment of the present disclosure.

### DETAILED DESCRIPTION OF ILLUSTRATIVE EMBODIMENTS

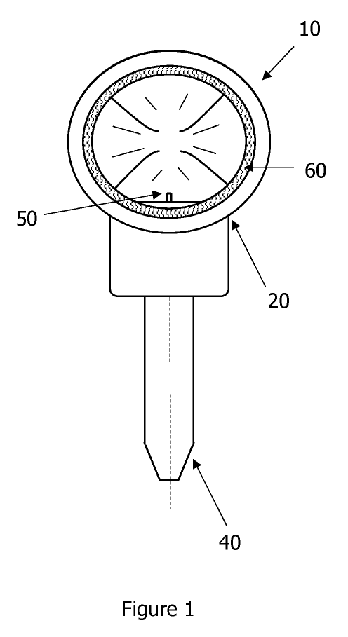

Referring to FIG. 1, embodiments of the present disclosure include a blood collection device 10. The blood collection device, also referred to as a collection device in this application, includes a housing 20, at least one blood collection container 40, at least one lancet 50 configured to pierce tissue, such as skin tissue of a human, and an absorbent material 60 integrated with the housing 20. The absorbent material 60 is configured to absorb fluids emanating from the tissue after the lancet 50 pierces the tissue, as further described below. The blood collection device 10 is configured to extract small amounts of blood from the patient for analysis. In this case, the blood collection device 10 is configured to extract micro samples of blood from the patient.

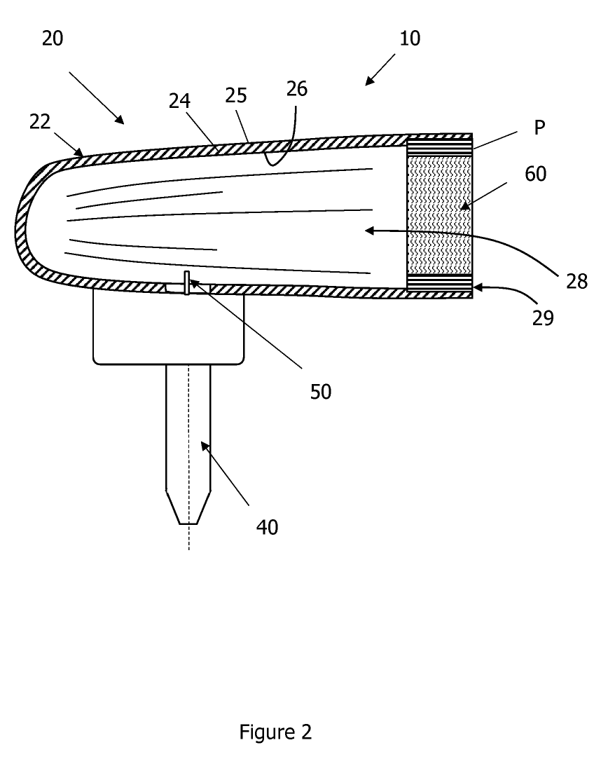

Referring to FIGS. 1 and 2, the housing 20 is designed for ergonomic fit in a user's hand while also being design fit around the patient's finger. The housing 20 includes a housing body 22 with an outer wall 24 that includes an outer surface 25 and an inner surface 26. The inner surface 26 may define a skin contact surface. Furthermore, the inner surface 26 defines an internal volume 28 that is dimensioned to receive therein a finger of the patient from whom blood is being collected. The outer edge 29 of the housing 22 defines an outer perimeter P and is also configured to face or contact the patient's skin in use. The housing 20 also includes an interface (not shown) and an actuator (not shown) for the lancet 50. The actuator is described further below. The interface is structured to receive and hold the blood collection containers 40. The interface may include features to encourage flow of blood from the skin to the blood collection container by routing the blood through a channel from the skin to the blood collection container after the lancet pierces the skin. A typical interface may be used.

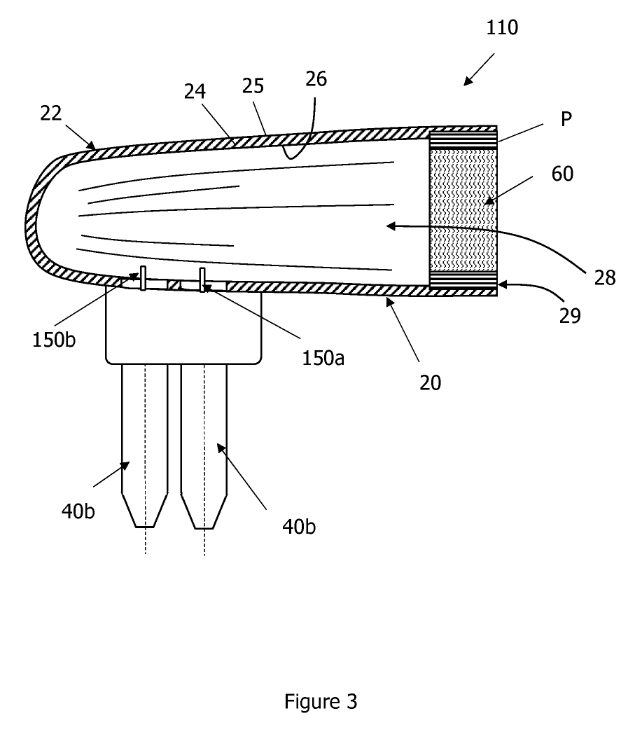

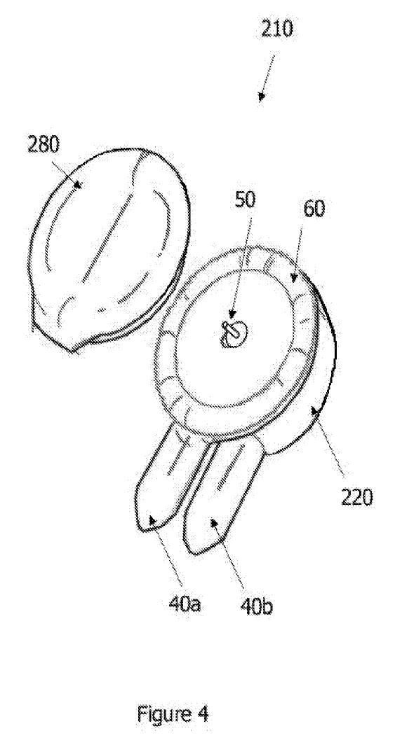

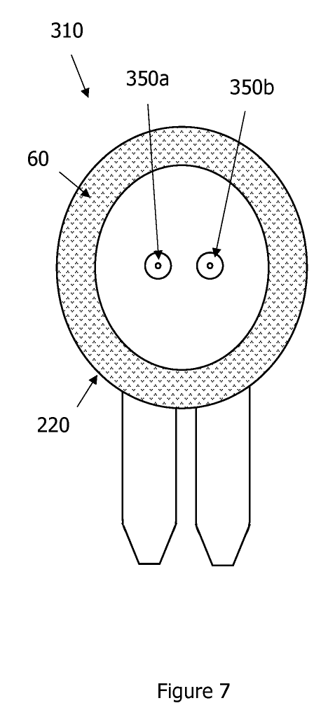

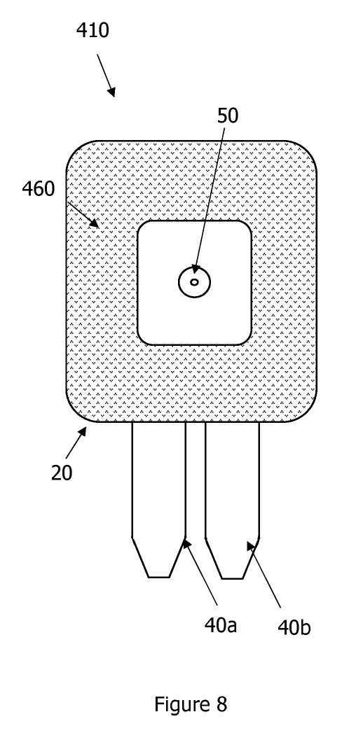

Referring to FIGS. 1 and 2, the illustrated device includes a lancet 50 configured to pierce tissue. The lancet 50 may be coupled to the interface in the housing 20. The lancet 50 may also extend from, or is configured to extend from the skin contact surface 26 into the internal volume 29 via operation of the actuator. The lancet 50 is this regard is a sharp instrument with a pointed tip designed to pierce tissue. In alternative embodiments, more than one lancet may be included, as illustrated in the blood collection device 110 shown in FIG. 2 and the blood collection devices shown in FIGS. 3-7. For clarity of illustration and description, the blood collection devices 10 and 110 shown in FIGS. 2 and 3 have similar reference signs to identify elements in common with the blood collection device 10 shown in FIGS. 1-2. In FIG. 3, the blood collection device 110 includes a pair of lancets 150a and 150b that extend from or are extendable into the inner volume 29 of the blood collection device 10. While embodiments with two lancets are shown, the blood collection devices as used herein may include more than two lancets. For instance, three lancets, four lancets, and more may be used. Furthermore, the blood collection device 210 shown in FIGS. 4-6, the blood collection device 310 shown in FIG. 7, and the blood collection device 410 shown in FIG. 8 may include a pair of lancets, as will be further explained below.

Referring to FIGS. 1-2, the blood collection device 10 also includes an actuator (not shown) configured to control the position of the lancet 50. In one example, the actuator is configured to cause the at least one lancet 50 to move between a retracted position, where the at least one lancet 50 is withdrawn in the housing 20, and an extended position, where the at least one lancet 50 extends into the inner volume 29 of the housing 20. After going from retracted position to the extended position, the lancet 50 moves back into the retracted position so that there is no prick hazard. The actuator may be operated via push button, lever, switch, or rotatable knob that may be used to cause the actuator to move the lancet 50 between the desired configurations. Any suitable actuator or mechanism that allows for the controlled movement of the lancet 50 between the extended and retracted configurations may be used.

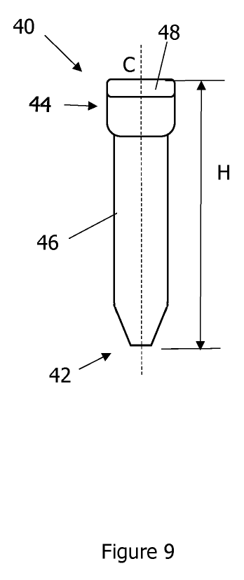

Continuing with FIGS. 1-2, the blood collection device 10 includes at least one blood sample collection container 40 coupled to the housing 20. The housing 20 is adapted to hold the blood sample collection container 40 in communication with the interface. The blood collection container 40 is sized to hold the blood sample once the lancet 50 pierces the tissue. Turning to FIG. 9, the blood collection container 40 is configured to hold a sample of blood and to engage the interface of the blood collection devices described herein. The blood collection container 40 has a lower end 42, an upper end 44, and a sidewall 46 that extends from the lower end 42 to the upper end 44. The blood collection container 40 extends along a central axis C, and has a height H that extends from the lower end 42 to the upper end 44 along the central axis C. The blood collection container 40 defines an internal volume that holds the blood sample. As discussed above, in one example, the internal volume is sufficient to hold between 0.5 to 1000 μl of blood. In one example, the volume is between 300 and 600 μl. In another example, the volume is between 200 and 300 μl. For instance, the sample container 90 is sized to hold a micro-sample. However, in other embodiments, the blood collection container 40 is sized to hold larger amounts of blood. As shown, the blood collection container 40 includes a cap 48 that can close off the upper end 44 of the blood collection container 40 and engage the blood collection device 10 at the interface (not shown). The blood collection container 40 may be coupled to the housing with the cap 48. In one example the blood collection container 40 is removable from the interface with the cap 48. Alternatively, the blood collection container 40 is removable without the cap 48 thereby leaving the cap in the housing. The blood collection container 30 may be at least partially transparent. In one example, the blood collection container 40 may be formed of glass. In another example, the blood collection container 40 is a polymeric material.

Turning back to FIG. 3, the blood collection device 110 according the alternative embodiment is illustrated as having a pair of blood collection containers 40. For instance, the blood collection device can hold a first blood collection container 40a and a second blood collection container 40b with the first blood collection container adapted for a first type of test and the second blood collection container adapted for a second type of test. In other words, the first blood collection container and the second blood collection container are different from each other. The reference signs 40, 40a, and 40b may be used interchangeably in this application. In some cases, the first and second blood collection containers 40a and 40b may have different additives or other agents that initiate clotting or other mechanism in the blood sample that aids analysis of the blood samples contained therein. In other embodiments, however, the blood collection device 10 may have a single blood collection container. In still other embodiments (not shown), the blood collection device 10 may have more than two blood collection containers. Even though the blood collection device 10 can include more than one or two blood collection containers, the interface is designed so that one only blood collection container may be used at a time if needed.

Turning back to FIGS. 1-2, the blood collection device 10 includes an absorbent material 60 along the inner surface 26 of the housing 20. As illustrated, the absorbent material 60 is integrated with the housing along the outer edge 29 of the housing 20. The absorbent material 60 is also substantially located on the inner surface 26 so as to be within the inner volume 28 of the housing 20. As shown, the absorbent material is a ring shape that extends around the entire edge 29 of the housing 20. However, the absorbent material 60 may be configured to extend around a portion of the inner surface 26 of the housing 20 along its edge 29. Alternatively, the absorbent material 60 can be disposed further into the housing 20 spaced away from the outer edge 29. In the illustrated embodiment, the absorbent material is adhesively bonded to the housing 20. However, in other configurations, the absorbent material may be mechanically attached to the housing 20. The absorbent material 60 is designed to absorb fluids. In this regard, the absorbent material may be formed from any absorbent component or structure. In one example, the absorbent material is knit, a woven, a nonwoven fabric material, or a combination thereof. Furthermore, the absorbent material 60 comprises cotton fibers, regenerated cellulose fibers, or a blend of cotton fibers and regenerated cellulose fibers. The location of the absorbent material 60 allows the patient to insert their finger into the housing 20. The lancet 50 is actuated to pierce the tissue and then retracted. As the patient removes his/her finger from the housing 20, the absorbent material 60 absorbs the blood and any other fluids that are released. This aids in clean up and reduce complexity of the collection process.

Referring to FIG. 4, an alternative embodiment of a blood collection device 210 is shown. For clarity of illustration and description, the blood collection device 210 shown in FIGS. 4-6 have similar reference signs to identify elements in common with the blood collection device 10 shown in FIGS. 1-2. As shown, the blood collection device 220 includes a housing 220, at least one blood collection container 40, at least one lancet 250 configured to pierce tissue, such as skin tissue of a human, and an absorbent material 60 integrated with the housing 220. The absorbent material 60 is configured to absorb fluids emanating from the tissue after the lancet 50 pierces the tissue, as further described below. The blood collection device 210 may include an optional cap 280 to cover the lancet region and absorbent material. The blood collection device 210 is configured to extract small amounts of blood from the patient for analysis. In this case, the blood collection device 210 is configured to extract micro samples of blood from the patient.

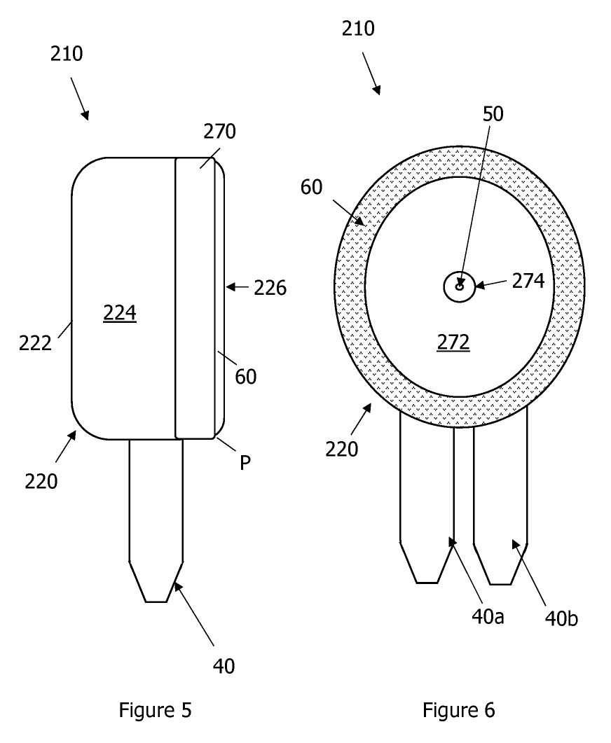

Referring to FIGS. 4 and 5, the housing 220 is designed for ergonomic fit in a user's hand. The housing includes a housing body 222 with an outer wall2 24 and a skin contact side 226. The skin contact side 226 defines an outer perimeter P and is also configured to face or contact the patient's skin in use. The housing 220 defines an internal space (not numbered) that includes an interface (not shown) and an actuator (not shown) for the lancet 50. The actuator is similar to the actuator described above used in device 10. The interface is structured to receive and hold the blood collection containers 40 and also similar to the interface described above with respect device 10. In this embodiment, the housing 220 is design to lie against a user's arm, abdomen, or legs such that skin contact side 226 is exposed for use.

As shown in FIGS. 4 and 6, the collection device 210 includes a single lancet. In alternative embodiments, more than one lancet may be included as illustrated in the blood collection device 310 shown in FIG. 7. For clarity of illustration and description, the blood collection device 310 shown in FIG. 7 has similar reference signs to identify elements in common with the blood collection device 10 shown in FIGS. 1-2 and blood collection device 210 shown in FIGS. 4-6. In FIG. 7, the blood collection device 310 includes a pair of lancets 350a and 350b that extend from or are extendable from the skin contact side 226 of the blood collection device 310. While embodiments with two lancets are shown, the blood collection devices as used herein may include more than two lancets.

Referring back to FIGS. 4-6, as discussed above, the blood collection device 210 also includes an actuator configured to control the position of the lancet 50. In one example, the actuator is configured to cause the at least one lancet 50 to move between a retracted position, where the at least one lancet 50 is withdrawn in the housing 220, and an extended position, where the at least one lancet 50 extends outward from the skin contact side 226 of the housing 220. After going from retracted position to the extended position, the lancet has to go back to the retracted position immediately so that there is no prick hazard. As discussed above, any suitable actuator or mechanism that allows for the controlled movement of the lancet 50 between the extended and retracted configurations may be used. For example, the actuator may be operated via push button, lever, switch, or rotatable knob.

Referring to FIGS. 4-6, the blood collection device 210 is illustrated as having a pair of blood collection containers 40. For instance, the blood collection device can hold a first blood collection container 40a and a second blood collection container 40b with the first blood collection container adapted for a first type of test and the second blood collection container adapted for a second type of test. As with other embodiment of the blood collection device described herein, therefore, the first blood collection container and the second blood collection container are different from each other. For instance, the first and second blood collection containers 40a and 40b may have different additives or other agents that initiate clotting or other mechanism in the blood sample that aids analysis of the blood samples contained therein. In other embodiments, however, the blood collection device 10 may have a single blood collection container. In still other embodiments (not shown), the blood collection device 10 may have more than two blood collection containers. Even though the blood collection device 10 can include more than one or two blood collection containers, the interface is designed so that one only blood collection container may be used at a time if needed.

Turning back to FIGS. 4-6, the blood collection device 210 includes an absorbent material 260 on the skin contact side 226 of the housing 220. As shown in FIGS. 4-6, the absorbent material 60 is attached to the housing 20 with a clamp 270 and securing plate 272. The securing plate 272 has an opening 274 through which the lancet 50 extends. The absorbent material 60 can extend over the skin contact side 26 with the clamp 270 either screwed onto the housing or otherwise mechanically fastened to the housing 220. The securing plate 272 is attached to the housing 20 such that the lancet 50 is aligned with the opening 274. The securing plate 272 may be screwed to the housing 20 or otherwise mechanically fastened to the housing 20. In this configuration, the absorbent material 60 defines a ring shape around the lancet 50 and also extends along the perimeter P of the blood collection device 10. The housing 20, clamp 270, and plate 272 may have other configurations so that the absorbent material 60 defines other shapes on the blood collection device 10. For example, in accordance with an alternative embodiment shown in FIG. 8, a blood collection device 410 may have an absorbent material 460 with a rectilinear shape. The blood collection device 410 is similar to the blood collection device 210 and the same references are used to illustrate elements of the blood collection device common between the two. It should be appreciated that other shapes, such as polyhedral shapes, may be used. Furthermore, the blood collection device 10 may use either a clamp 270 or the plate 272 to secure the absorbent material 60 to the housing 20. In still other embodiments, the absorbent material is adhesively bonded to the housing 20.

In using the blood collection devices 10 and 110 illustrated in FIGS. 1-3, the blood collection device 10, 110 is inserted over the patient's finger so that the finger is within the internal volume 28 of the housing 20. The lancet (s) 50 is actuated to extend into the tissue to pierce the tissue and then retracted back again. The interface draws and/or guides blood in the respective collection containers 40. The collection device 10, 110 is then removed from the finger causing the absorbent material to wipe off the blood and fluid from the pierced area. Thus, the blood collection device 10, 110 itself can be used to absorb any blood near the piercing site in contrast to conventional blood collection devices.

In accordance with the alternative embodiments illustrated in FIGS. 3-8, the blood collection devices 210, 310, 410, the collection technician can hold the blood collection device 210, 310, 410 near the target site of the patient. The lancet (s) 50 is actuated to extend into the tissue to pierce the tissue and then retracted back again. The interface draws and/or guides blood in the respective collection containers 40. The collection device 210, 310, 410 is then dragged over the piercing and the fluid is absorbed. Thus, the blood collection device 210, 310, 410 itself can be used to absorb any blood near the piercing site in contrast to conventional blood collection devices.

Conventional blood collection techniques use a separate gauze pad which is contained in its own sterile packaging. By integrating the absorbent material into the blood collection devices described above the steps to collect the blood are reduced and simplified. In conventional devices and methods, one common solution to the multitasking problem discussed above is to ask the patient to assist in the collection process. Collection technicians will often ask the patient to apply gauze to themselves while the collection technician completes inverting the final tube. The blood collection devices described herein allow the user to apply absorbent material to the patient after blood collection is completed. In this case, the user may use the same hand to both collect blood and cleanup the puncture site from the lancet.

While the present disclosure is described herein using a limited number of embodiments, these specific embodiments are not intended to limit the scope of the disclosure as otherwise described and claimed herein. Modification and variations from the described embodiments exist. It should be understood that the invention is not limited to the specific details set forth in the examples.

1. A blood collection device, comprising:
a housing having an interface;
at least one blood collection container engaged with the interface;
at least one lancet coupled to the interface and extending or configured to extend from the interface, the at least one lancet configured to pierce tissue; and
an absorbent material integrated with the housing, the absorbent material configured to absorb fluids emanating from the tissue after the at least one lancet pierces the tissue.

2. The blood collection device of claim 1, wherein the housing has an inner surface that defines an inner space for receiving a finger, wherein the absorbent material disposed along the inner surface.

3. The blood collection device of claim 2, wherein the at least one lancet is configured to extend into the inner volume of the housing.

4. The blood collection device claim 1, wherein the housing is sized to hold at least a portion of the at least blood collection container.

5. The blood collection device of claim 1, wherein the housing defines a skin contact side, and the absorbent material is disposed on the skin contact side of the housing.

6. The blood collection device of claim 1, wherein the skin contact side defines an outer perimeter, wherein the absorbent material is disposed at least partially along the outer perimeter.

7. The blood collection device of claim 1, wherein the absorbent material defines a ring shape around the housing.

8. The blood collection device of claim 1, wherein the absorbent material has one of circular shape, a rectilinear shape, or polyhedral shape.

9. The blood collection device of claim 1, wherein the absorbent material is bonded to the housing.

10. The blood collection device of claim 1, wherein the absorbent material is attached to the housing with a clamp.

11. The blood collection device of claim 1, wherein the absorbent material is knit, a woven, or a nonwoven fabric material.

12. The blood collection device of claim 1, wherein the absorbent material comprises cotton fibers, regenerated cellulose fibers, or a blend of cotton fibers and regenerated cellulose fibers.

13. The blood collection device of claim 1, further comprising an actuator configured to cause the at least one lancet to move between a retracted position, where the at least one lancet is withdrawn in the housing, and an extended position, where the at least one lancet extends outward from the skin contact side of the housing.

14. The blood collection device of claim 1 wherein the at least one lancet is two lancets.

15. The blood collection device of claim 1, wherein the at least one blood collection container is a first blood collection container and a second blood collection container.

16. The blood collection device of claim 15, wherein the first blood collection container and the second blood collection container are different.

17. The blood collection device of claim 1, wherein the at least one blood collection container is coupled to the housing with the cap.

18. The blood collection device of claim 17, wherein the at least one blood collection container is removable from the housing with the cap or without the cap leaving the cap coupled to the housing.

19. The blood collection device of claim 16, wherein the at least one lancet is a first lancet associated with the first blood collection container and a second lancet associated with the second blood collection container.

20. A blood collection device, comprising:
a housing having an outer surface, an inner surface opposite the outer surface, and internal space defined by the inner surface, and an interface, the inner surface defining a skin contact portion;
at least one blood collection container engaged with the interface;
at least one lancet coupled to the interface and extending or configured to extend from the interface into the internal space of the housing, the at least one lancet configured to pierce tissue; and
an absorbent material integrated with the housing, the absorbent material configured to absorb fluids emanating from the tissue after the at least one lancet pierces the tissue.

21. The blood collection device of claim 20, wherein the at least one lancet is configured to extend into the inner volume of the housing.

22. The blood collection device of claim 20, wherein the housing is sized to hold at least a portion of the at least blood collection container.

23. The blood collection device of claim 20, wherein the housing defines an outer perimeter, wherein the absorbent material is disposed at least partially along the outer perimeter.

24. The blood collection device of claim 20, wherein the absorbent material defines a ring shape around the housing.

25. The blood collection device of claim 1, wherein the absorbent material is adhesively bonded to the housing.

26. The blood collection device of claim 20, further comprising an actuator configured to cause the at least one lancet to move between a retracted position, where the at least one lancet is withdrawn in the housing, and an extended position, where the at least one lancet extends outward from the skin contact side of the housing.

27. The blood collection device of claim 20, wherein the at least one lancet is two lancets.

28. The blood collection device of claim 20, wherein the at least one blood collection container is a first blood collection container and a second blood collection container.

29. The blood collection device of claim 20, wherein the first blood collection container and the second blood collection container are different.

30. The blood collection device of claim 20, wherein the at least one blood collection container is coupled to the housing with the cap.

31. The blood collection device of claim 30, wherein the at least one blood collection container is removable from the housing with the cap or without the cap leaving the cap coupled to the housing.
